(19) United States
(12) Patent Application Publication
(10) Pub. No.: US 2014/0378699 A1
(43) Pub. Date: Dec. 25, 2014

(54) PROCESS FOR THE PREPARATION OF FESOTERODINE

(71) Applicant: ALEMBIC PHARMACEUTICALS LIMITED, Vadodara (GJ) (IN)

(72) Inventors: Venkat Raman Jayaraman, Vadodara (IN); Samir Patel, Vadodara (IN); Indrajit Thakor, Vadodara (IN); Viral Maheshbhai Parekh, Vadodara (IN); Mahesh Ladani, Vadodara (IN); Chetan Patil, Vadodara (IN); Ronak Patel, Vadodara (IN); Prashant Raval, Vadodara (IN)

(21) Appl. No.: 14/370,717

(22) PCT Filed: Jan. 4, 2013
(86) PCT No.: PCT/IB2013/050089

§ 371 Date: Jul. 3, 2014

(30) Foreign Application Priority Data
Jan. 7, 2012 (IN) 62/MUM/2012

(51) Int. Cl. C07C 209/68

(52) U.S. Cl. CPC C07C 209/68
USPC 560/238; 564/374

(57) ABSTRACT

The present invention relates to an improved process for the preparation of Fesoterodine and pharmaceutically acceptable salts thereof. The present invention particularly relates to a process for the preparation of Fesoterodine from O-benzyl tolterodine.

### FIELD OF THE INVENTION

The present invention relates to an improved, commercially viable and industrially advantageous process for the preparation of Fesoterodine or a pharmaceutically acceptable salt thereof in high yield and purity. More specifically the present invention relates to an improved and industrially advantageous oxidation method to convert O-benzyl tolterodine to Fesoterodine and salts thereof.

### BACKGROUND OF THE INVENTION

Fesoterodine is [2-[(1R)-3-(Di(propan-2-yl)amino)-1-phenylpropyl]-4-(hydroxymethyl) phenyl]2-methylpropanoate and represented by formula (I).

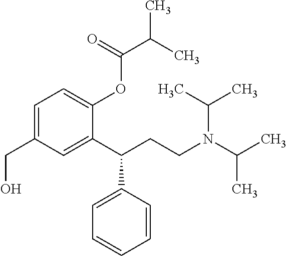 [SMILES: CC(C)C(=O)Oc1ccc(CO)cc1[C@H](CCN(C(C)C)C(C)C)c1ccccc1]

The product is marketed in the form of Fumarate salt. The current pharmaceutical product containing this drug is being sold by Pfizer using the trade name Toviaz, in the form of extended release oral tablets. Fesoterodine is cholinergic antagonist and muscarinic antagonist. Fesoterodine is rapidly de-esterified to its active metabolite, (R)-2-(3-diisopropylamino-1-phenylpropyl)-4-hydroxymethyl-phenol, or 5-hydroxy methyl tolterodine, which is a muscarinic receptor antagonist. Fesoterodine is used as Urinary Incontinence Products. It is used to treat overactive bladder.

Few processes for the synthesis of 3,3-diphenylpropylamine derivatives have been described in the literature. Tolterodine and other 3,3-diphenyl propylamine analogs were first described in U.S. Pat. No. 5,382,600. U.S. Pat. No. 6,713,464 disclosed a variety of 3,3-diphenylpropylamine derivatives, processes for their preparation, pharmaceutical compositions in which they are present and method of use thereof.

WO2005012227 describes process for preparation of Fesoterodine from Tolterodine. However, this process involves preparation of resolved Tolterodine and then its benzylation, increase the number of steps such as debenzylation to prepare tolterodine, resolution of tolterodine and then further benzylation to convert in R (+) benzyl tolterodine which further gets converted to Fesoterodine. Further this process involves oxidation of methyl group to aldehyde group using copper sulphate and sodium persulphate. However, during this conversion many impurities develop, reaction is very slow and unreacted material remains as such which reduces the overall yield and purity. Further, this process requires excess of metal persulphate which increase overall cost.

WO2012137047 describes process for preparation of Fesoterodine. In this process oxidation of O-benzyl tolterodine is carried out using ceric ammonium nitrate. The relative higher cost of ceric ammonium nitrate and the toxic effluent ingredients demand safer alternatives for this oxidation reaction.

Thus, a need still remains for an improved and commercially viable process of preparing pure Fesoterodine or a pharmaceutically acceptable salt thereof that will solve the aforesaid problems associated with process described in the prior art and will be suitable for large-scale preparation.

To address this need, a process has been developed using readily available oxidizing agent which is also environment friendly. In the present process use of metal persulphate in presence of coreductant achieves the intended transformation described below. This process gives assurance about the ease of scale up in commercial quantity.

### SUMMARY OF THE INVENTION

The present inventors have focused on the problems associated with the prior art process and has developed an improved process for the preparation of Fesoterodine.

Therefore, in one aspect, the present invention provides a process of oxidizing compound A in to compound B in presence of metal persulphate, organic or inorganic coreductant and optionally inorganic acid

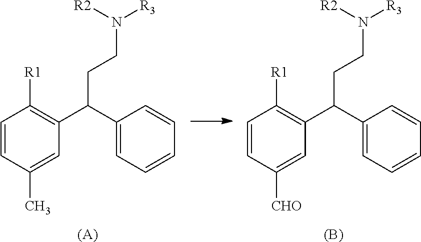

wherein,

R1 is hydrogen, hydroxy, alkyl, alkoxy, hydroxyalkyl, trifluoromethyl, disubstituted amino, nitro, alkylcarbonylamino, alkylcarbonyloxy, halogen,

R2 and R3 represent C1-C6 alkyl groups, which may be the same or different and which together contain at least three carbon atoms, or R2 and R3 may form a ring together with the amine nitrogen.

In another aspect, present invention provides a process of oxidizing compound A in to compound B in presence of metal persulphate, organic or inorganic coreductant and optionally inorganic acid

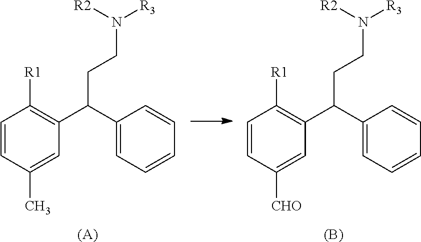

wherein,

R1 is hydrogen, hydroxy, alkyl, alkoxy, hydroxyalkyl, trifluoromethyl, disubstituted amino, nitro, alkylcarbonylamino, alkylcarbonyloxy, halogen,

R2 and R3 represent C1-C6 alkyl groups, which may be the same or different and which together contain at least three carbon atoms, or R2 and R3 may form a ring together with the amine nitrogen.

In another aspect, metal persulphate is selected from the group of sodium persulphate, ammonium persulphate or potassium persulphate more preferably sodium persulphate. In another aspect the coreductant is selected from dialkylsulphides, diaryl sulphides, alkyl aryl sulphides, dialkylsulfoxides, diarylsulfoxides, alkylarylsulfoxides, mono or poly substituted amines, mono or poly substituted phosphines; more preferably, the coreductant is selected from dimethyl sulphoxide or dimethyl sulphide.

In another aspect, the inorganic acid is selected from the group of sulphuric acid, hydrochloric acid and nitric acid.

In another aspect, present invention provides a process of oxidizing compound A in to compound B in presence of sodium persulphate, dimethyl sulphoxide and optionally with sulphuric acid.

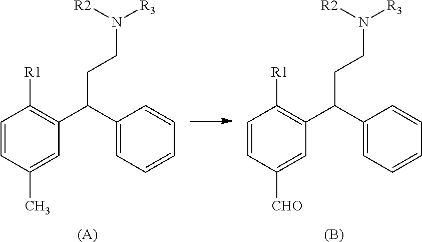

wherein,

R1 is hydrogen, hydroxy or substituted or unsubstituted alkyl, aryl, aryloxy, alkoxy, hydroxyalkyl, trifluoromethyl, disubstituted amino, nitro, alkylcarbonylamino, alkylcarbonyloxy, halogen, arylalkyloxy, substituted silyloxy,

R2 and R3 represent C1-C6 alkyl groups, which may be the same or different and which together contain at least three carbon atoms, or R2 and R3 may form a ring together with the amine nitrogen.

In another aspect, present invention provides a process for preparing Fesoterodine or its enantiomer, or a salt thereof, comprising a step of oxidizing a compound of formula II or its enantiomer in to compound of formula III or its enantiomer in presence of sodium persulphate, dimethyl sulphoxide and optionally with sulphuric acid.

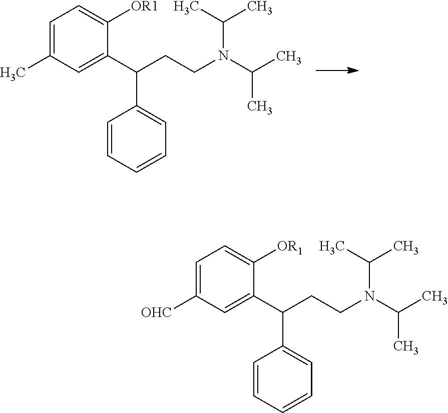

Where R1 is any hydroxyl protecting group,

In another aspect, present invention provides a process for preparing Fesoterodine or its enantiomer, or a salt thereof, comprising a step of oxidizing a compound of formula II or its enantiomer in to compound of formula III or its enantiomer using metal sulphate, metal acetate and metal persulphate in presence of inorganic acid and coreductant.

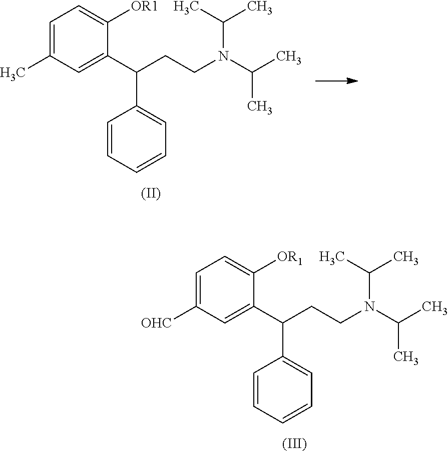

Where R1 is any hydroxyl protecting group,

In another aspect, metal sulphate is selected from the group of copper sulphate, ferrous sulphate, zinc sulphate, magnesium sulphate, manganese sulphate, more preferably ferrous sulphate.

In another aspect, metal acetate is selected from the group of sodium acetate, potassium acetate, magnesium acetate, calcium acetate, nickel acetate, copper acetate, zinc acetate, iron acetate, more preferably copper acetate.

In another aspect, present invention provides a metal persulphate is selected from the group of sodium persulphate, ammonium persulphate or potassium persulphate more preferably sodium persulphate.

In another aspect, an inorganic acid is selected from the group of sulphuric acid, hydrochloric acid, hydrobromic acid or hydroiodic acid.

In another aspect, present invention provides coreductant is selected from dimethyl sulphoxide or dimethyl sulphide.

In another aspect, present invention provides a process for preparing Fesoterodine or its enantiomer, or a salt thereof, comprising a step of oxidizing compound of formula IV to convert in compound of formula V using sodium persulphate, copper sulphate, copper acetate and sulphuric acid in presence of dimethyl sulphoxide.

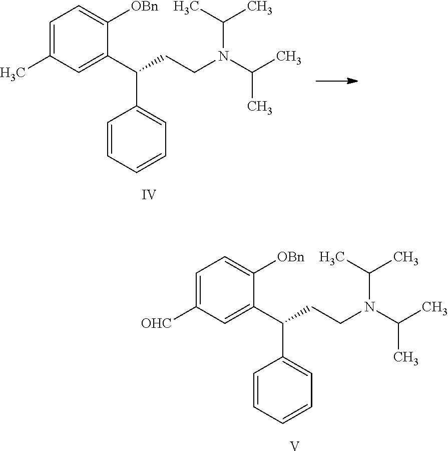

In another aspect, coreductant is selected from dimethyl sulphoxide or dimethyl sulphide.

The present inventors have found that employing process of the present invention for the preparation of Fesoterodine, overcomes the drawbacks of the prior art and may be prepared and subsequently converted to Fesoterodine in high yield and purity.

### DETAILED DESCRIPTION OF THE INVENTION

The present invention provides a process for preparation of Fesoterodine or a pharmaceutically acceptable salt thereof, which comprises:

The process for preparation of Fesoterodine Fumarate is shown in the scheme I.

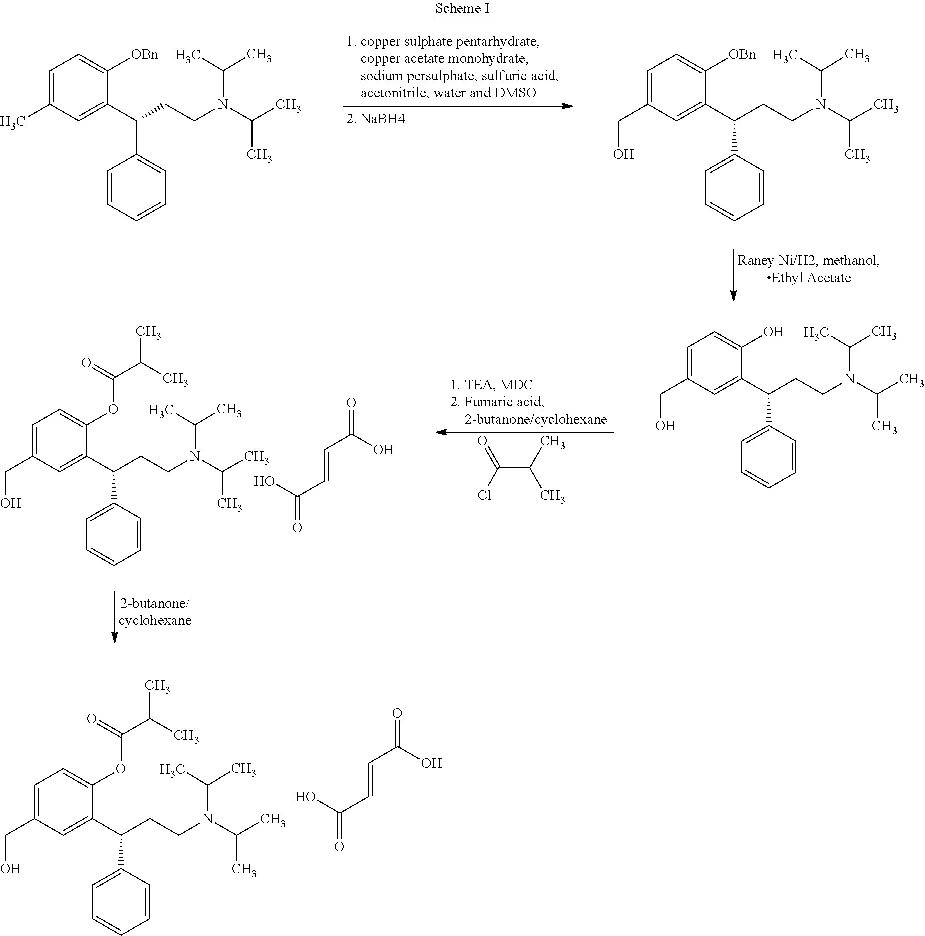

In one embodiment of the present invention provides a process for preparing Fesoterodine or its enantiomer, or a salt thereof, comprising a step of oxidizing a compound of formula II or its enantiomer in to compound of formula III or its enantiomer using metal sulphate, metal acetate and metal persulphate in presence of inorganic acid and organic solvents optionally with water.

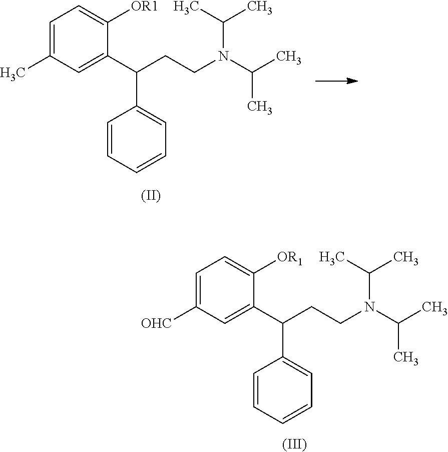

Where R1 is any hydroxyl protecting group,

Metal sulphate is selected from the group of copper sulphate, ferrous sulphate, zinc sulphate, magnesium sulphate, manganese sulphate, more preferably ferrous sulphate, coreductant is selected from dialkylsulphides, diaryl sulphides, alkyl aryl sulphides, dialkylsulfoxides, diarylsulfoxides, alkylarylsulfoxides, mono or poly substituted amines, mono or poly substituted phosphines; more preferably, the coreductant is selected from dimethyl sulphoxide or dimethyl sulphide.

Metal acetate is selected from the group of sodium acetate, potassium acetate, magnesium acetate, calcium acetate. Magnesium acetate, nickel acetate, copper acetate, zinc acetate, iron acetate, more preferably copper acetate. Metal Persulphate is selected from the group of sodium persulphate, Sodium peroxomonosulfate, Sodium peroxodisulfate, ammonium persulphate or potassium persulphate more preferably sodium persulphate. Organic solvents are selected from the group of dimethyl formamide, chloroform, acetonitrile, tetrahydrofuran, toluene, dimethyl acetamide or mixtures thereof. Inorganic acid is selected from the group of sulphuric acid, hydrochloric acid, hydrobromic acid or hydroiodic acid.

Wherein hydroxyl protecting group are selected from the group of Acetyl, Benzoyl, substituted Benzoyl, Benzyl, substituted benzyl, Dimethoxy trityl, Methoxy trityl, and the like, more preferably Benzyl.

The method of the invention makes use of persulphate, which is a stable compound which is not very toxic and has a good oxidizing power.

Therefore, in one embodiment the present invention provides a process of oxidizing compound A in to compound B in presence of metal persulphate, organic or inorganic coreductant and optionally inorganic acid,

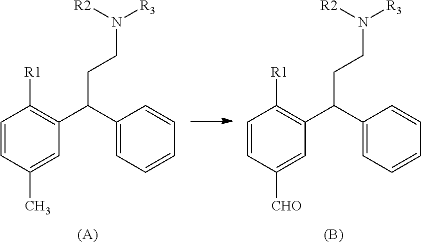

wherein, R1 is hydrogen, hydroxy or substituted or unsubstituted alkyl, aryl, aryloxy, alkoxy, hydroxyalkyl, trifluoromethyl, disubstituted amino, nitro, alkylcarbonylamino, alkylcarbonyloxy, halogen, arylalkyloxy, substituted silyloxy,

R2 and R3 represent C1-C6 alkyl groups, which may be the same or different and which together contain at least three carbon atoms, or R2 and R3 may form a ring together with the amine nitrogen.

More specifically present invention provides a process as shown in scheme-II

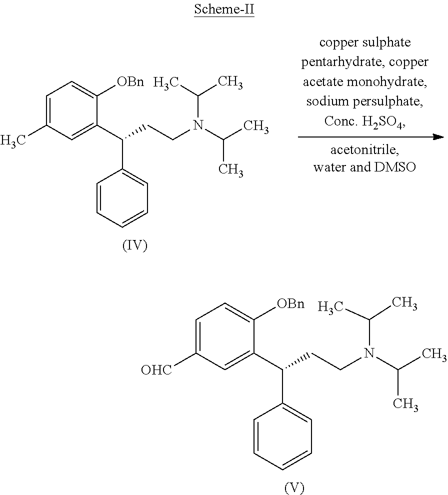

The process of the present invention has following advantages:

1. Less impurity formation.

2. Product obtained is better in terms of yield and purity, compared to other processes.

3. Generates effluent which can be handled easily.

The present invention further illustrated in detail by the below examples which are however not limit to the scope of the invention.

### EXAMPLES

### Example-1

### Preparation of R (+)-[4-benzyloxy-3-(3-diisopropylamino-1-phenyl-propyl)-phenyl]-methanol

Charged R-(+)-N,N-diisopropyl-3-(2-benzyloxy-5-methylphenyl)-3-phenyl propane amine (51.0 g), Acetonitrile (500 ml), Process water (400 ml) and DMSO (100 ml) into the reaction mass and stirred the reaction mass at 30±35° C. Slowly added cone, sulphuric acid (2.0 gm) and stirred the reaction mass at 30±35° C. Charged Copper acetate monohydrate (4.13 g), Ferrous sulfate heptahyrate (13.86 g) and Sodium persulfate (59.36 g) at 30±5° C. The contents were stirred at 80° C. for 1 hr followed by further addition of Copper acetate monohydrate (1.02 g), Ferrous sulfate heptahyrate (5.02 g) and Sodium persulphate (17.79 g) at 60° C. Maintained at 80° C. till completion of the reaction. Added 10% aqueous solution of Sodium hydroxide (275 ml) to adjust pH 6.0 to 7.0 between 0 to 5° C. followed by lot wise addition of Sodium Borohydride (14.18 g) at 5±3° C. After completion of the reaction Conc. Hydrochloric acid was added and stirred the reaction mass for 90-120 min at 35±3° C. The product was extracted in Dichloromethane (200 ml) and distilled out Dichloromethane completely to give 42.3 g R-(+)-[4-benzyloxy-3-(3-diisopropylamino-1-phenyl-propyl)-phenyl]-methanol. Yield 78.5%.

### Example-2

### Preparation of R (+)-2-(3-diisopropylamino-1-phenylpropyl)-hydroxymethyl phenol

R-(+)-[4-benzyloxy-3-(3-diisopropylamino-1-phenyl-propyl)-phenyl]-methanol (65.0 g), Raney Nickel (40 g) and Methanol (100 ml) were taken in to a hydrogenator and Maintained 4-5 Kg/Cm pressure of Hydrogen for 180-240 min. The mixture was then filtered and the solvent was removed by vacuum at below 50° C. The residue was crystallized in ethyl acetate (60 ml) to give 37.0 g R-(+)-[4-hydroxy-3-(3-diisopropylamino-1-phenyl-propyl)-phenyl]-methanol. Yield 72.5%.

### Example-3

### Preparation of R-(+)-2-(3-Diisopropylamino-1-phenylpropyl)-4-hydroxy methyl-phenyl isobutyrate ester hydrogen fumarate

A solution of R-(+)-2-(3-diisopropylamino-1-phenylpropyl)-4-hydroxymethylphenol (100.0 g) and triethylamine (30.0 g) in 1200 ml dichloromethane has a solution of isobutyryl chloride (34.0 g) in 500 ml dichloromethane added under agitation and cooling. Following addition agitation takes place for a further 15 minutes at 0° C., then for 30 minutes at room temperature. After completion of the reaction, reaction mass was washed with water (250 ml) and 5% aqueous sodium hydrogen carbonate solution. The organic phase is separated and concentrated on the rotary evaporator until dry. Obtained ester was dissolved in 2-butanone (300 ml) and then added fumaric acid (34.0 g). Complete dissolution of the acid on slight heating. Cyclohexane (75 ml) was slowly added under agitation until the onset of turbidity. The reaction mass was stirred for 6-7 hrs at room temperature and then at 3±3° C. for 60-90 min. The reaction mass was filtered and washed with cold (150 ml) Methylethylketone: Cyclohexane mixture (75:25) at 3±3° C. The wet cake was dried at 30±3° C. under vacuum. Yield 71%.

### Example-4

### Purification of Fesoterodine Fumarate

Methylethylketone (200 ml) and crude Fesoterodine Fumarate (100.0 g) was charged in to RB Flask and warm the reaction mass at 43±2° C. to get clear solution. Cyclohexane (62.5 ml) was added to the reaction mass and reaction mass was stirred for 720-840 min at 25±2° C. The reaction mass was cooled to 3±3° C. for 60-90 min and then filtered it. The reaction mass was washed with cold (150 ml) Methylethylketone: Cyclohexane mixture (75:25) at 3±3° C. and then with Cyclohexane (200 ml) at 25±3° C. The wet cake was dried at 30±3° C. under vacuum. Yield 90%.

1. A process of oxidizing compound A in to compound B in presence of metal persulphate, organic or inorganic coreductant and optionally with inorganic acid, 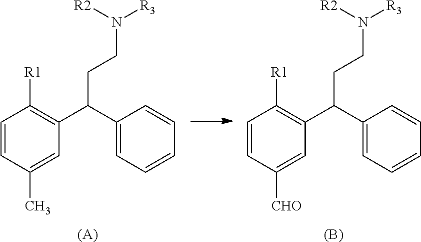
wherein R1 is hydrogen, hydroxy or substituted or unsubstituted alkyl, aryl, aryloxy, alkoxy, hydroxyalkyl, trifluoromethyl, disubstituted amino, nitro, alkylcarbonylamino, alkylcarbonyloxy, halogen, arylalkyloxy, or substituted silyloxy, and
R2 and R3 represent C1-C6 alkyl groups, which may be the same or different and which together contain at least three carbon atoms, or R2 and R3 may form a ring together with the amine nitrogen.

2. A process according to claim 1, wherein the metal persulphate is selected from the group consisting of sodium persulphate, ammonium persulphate or potassium persulphate.

3. A process according to claim 1, wherein said coreductant is selected from the group consisting of dialkylsulphides, diaryl sulphides, alkyl aryl sulphides, dialkylsulfoxides, diarylsulfoxides, alkylarylsulfoxides, mono or poly substituted amines, and mono or poly substituted phosphines.

4. A process according to claim 3, wherein said coreductant is dimethyl sulphoxide or dimethyl sulphide.

5. The process according to claim 1, wherein inorganic acid is selected from the group consisting of sulphuric acid, hydrochloric acid, hydrobromic acid and hydroiodic acid.

6. A The process of claim 1 wherein the metal persulphate is sodium persulphate, the organic coreductant is dimethyl sulphoxide and the optional inorganic acid is sulphuric acid.

7. (canceled)

8. A process for preparing Fesoterodine or its enantiomer, or a salt thereof, comprising a step of oxidizing a compound of formula II or its enantiomer in to compound of formula III or its enantiomer using metal sulphate, metal acetate and metal persulphate in presence of inorganic acid and coreductant. 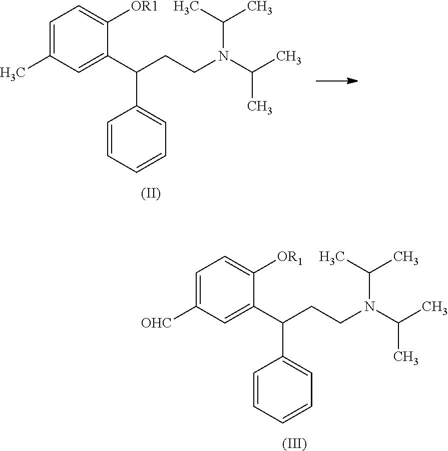
where R1 is any hydroxyl protecting group.

9. The process according to claim 8, wherein the metal sulphate is selected from the group consisting of copper sulphate, ferrous sulphate, zinc sulphate, magnesium sulphate, and manganese sulphate.

10. The process according to claim 8, wherein metal acetate is selected from the group consisting of sodium acetate, potassium acetate, magnesium acetate, calcium acetate, magnesium acetate, nickel acetate, copper acetate, zinc acetate, and iron acetate.

11. The process according to claim 8, wherein the metal persulphate is selected from the group consisting of sodium persulphate, ammonium persulphate and potassium persulphate.

12. The process according to claim 8, wherein the inorganic acid is selected from the group consisting of sulphuric acid, hydrochloric acid and nitric acid.

13. A process according to claim 8, wherein said coreductant is dimethyl sulphoxide or dimethyl sulphide.

14. A process for preparing Fesoterodine or its enantiomer, or a salt thereof, comprising a step of oxidizing compound of formula IV 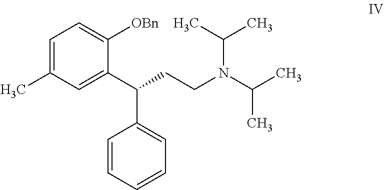 [SMILES: Cc1ccc(OCc2ccccc2)c([C@H](CCN(C(C)C)C(C)C)c2ccccc2)c1]
to convert in to compound of formula V 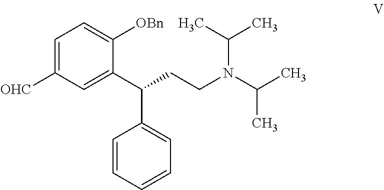 [SMILES: CC(C)N(CC[C@H](c1ccccc1)c1cc(C=O)ccc1OCc1ccccc1)C(C)C]
using sodium persulphate, copper sulphate, copper acetate and sulphuric acid in presence of dimethyl sulphoxide.

15. The process of claim 9, wherein the metal sulphate is ferrous sulphate.

16. The process of claim 10, wherein the metal acetate is copper acetate.

17. The process of claim 11, wherein the metal persulphate is sodium persulphate.
